(19) United States
(12) Patent Application Publication
(10) Pub. No.: US 2026/0199557 A1
(43) Pub. Date: Jul. 16, 2026

(54) COPPER-BASED MOF-MODIFIED SMALL-DIAMETER POLYURETHANE VASCULAR GRAFT AND PREPARATION METHOD THEREFOR

(71) Applicant: DALIAN UNIVERSITY OF TECHNOLOGY, Dalian (CN)

(72) Inventors: Xufeng DONG, Dalian (CN); Xijing ZHUANG, Dalian (CN); Huimin LIU, Dalian (CN); Chang XU, Dalian (CN)

(21) Appl. No.: 19/563,941

(22) Filed: Mar. 11, 2026

(30) Foreign Application Priority Data
Oct. 13, 2025 (CN) 202511453851.6

(51) Int. Cl. A61L 27/50; A61F 2/06; A61L 27/18; A61L 27/34; A61L 27/48; A61L 27/54; A61L 27/56

(52) U.S. Cl. CPC A61L 27/507; A61F 2/06; A61L 27/18; A61L 27/34; A61L 27/48; A61L 27/54; A61L 27/56; A61F 2230/0069; A61F 2250/0067; A61L 2300/102; A61L 2300/114; A61L 2300/414; A61L 2300/602; A61L 2420/06

(57) ABSTRACT

The present invention belongs to the technical field of biomedical composite materials, and discloses a copper-based MOF-modified small-diameter polyurethane vascular graft and a preparation method therefor. The present invention uses a copper-based metal organic framework as a functional carrier to be compounded with polyurethane, and uses electrospinning technology to prepare a vascular graft with an inner diameter of ≤6 mm, which achieves synergistic release of copper ions, nitric oxide and drug molecules. A preparation process includes the steps of copper-based metal organic framework synthesis, polyurethane solution preparation, drug loading, electrospinning molding, etc. The present invention innovatively constructs a synergistic system of “mechanical adaptation—biological regulation”, which effectively promotes the proliferation of endothelial cells while inhibiting the excessive proliferation of smooth muscle cells, and provides an ideal microenvironment for vascular repair. The present invention solves the clinical problems of mechanical property mismatch, thrombosis, endometrial hyperplasia, etc. of small-diameter vascular grafts.

### TECHNICAL FIELD

The present invention belongs to the technical field of biomedical composite materials, and relates to a copper-based MOF-modified small-diameter polyurethane vascular graft and a preparation method therefor.

### BACKGROUND

Cardiovascular diseases are leading noncommunicable fatal diseases worldwide, and vascular replacement surgery is a core treatment method for severe cardiovascular diseases. A vascular graft is a key implanted device in such surgery. At present, large-diameter vascular grafts made of polyethylene terephthalate, polytetrafluoroethylene, etc. have been applied in clinical practice on a large scale. However, clinical transformation of small-diameter vascular grafts is hindered due to the facts that the low-speed blood flow in the lumen after transplantation is prone to induce platelet aggregation and thrombosis, and the small-diameter vascular grafts have the technical bottlenecks such as low endothelialization efficiency, immunological rejection and mechanical property mismatch.

Metal-organic framework materials (MOFs), as porous crystal materials formed by the self-assembly of metal ions and organic ligands, have become a research focus in the fields of tissue engineering and regenerative medicine due to the structural designability and functional tunability. Compared with traditional artificial vascular materials, MOF-modified composite materials have porous structures and controllable surface chemical properties, which can achieve precise loading and controlled release of bioactive molecules. Relevant studies (Abolfazl Anvari Kohestani et al., Advanced Engineering Materials, 2023) show that zeolitic imidazolate framework-8 nanoparticles can effectively regulate the mechanical properties, drug release rate and drug release cycle of materials, and endow the materials with biological activities, thus providing a new strategy for solving the problems such as insufficient endothelialization and thrombosis in small-diameter vascular grafts.

At present, MOF materials still face key challenges in vascular tissue engineering applications: the balance between mechanical properties and biological activities of the materials, the spatiotemporal controllable release of multiple types of bioactive molecules, and the optimization of material surface characteristics to promote rapid endothelialization and inhibit excessive proliferation of smooth muscle cells. Future studies should focus on the long-term biocompatibility of materials, the kinetics of in vivo degradation and the integration mechanism with host tissues, thus to provide a theoretical basis and technical support for the development of next-generation small-diameter vascular grafts.

### SUMMARY

The purpose of the present invention is to provide a copper-based MOF-modified small-diameter polyurethane vascular graft and a preparation method therefor to solve the problems of thrombosis and endometrial hyperplasia caused by mechanical property mismatch and single biological function of small-diameter vascular grafts after in vivo implantation.

The technical solution of the present invention is as follows:

A copper-based MOF-modified small-diameter polyurethane vascular graft comprises a copper-based metal organic framework, polyurethane and drug molecules. Through electrospinning technology, the polyurethane is used as a base material for the small-diameter polyurethane vascular graft, and the drug molecules are loaded on the copper-based metal organic framework based on a porous structure thereof to modify the surface of the small-diameter polyurethane vascular graft, thus to endow the small-diameter vascular graft with the multi-level micro-regulation functions of “anti-thrombosis—endothelium promotion—hyperplasia inhibition”. The copper-based metal organic framework is self-assembled by metal centers and organic ligands through coordination bonds to form a porous crystal structure. Each metal center is a copper ion or a copper cluster provided by a copper source, and the copper source is selected from at least one of an inorganic acid salt, an organic acid salt, an oxide and a halide of copper; each organic ligand is an organic compound with at least two coordination loci, and the coordination loci are selected from at least one of carboxyl (—COOH), amino (—NH₂), hydroxyl (—OH), pyridyl (—C₅H₄N), imidazolyl (—C₃H₃N₂) or triazolyl (—C₂H₂N₃); and the drug molecules are vascular endothelial growth factor.

A preparation method for the copper-based MOF-modified small-diameter polyurethane vascular graft comprises the following steps:
Step 1: mixing tetrahydrofuran with N,N-dimethylformamide in a volume ratio, and adding polyurethane to prepare a solution A;
Step 2: adding gelatin into a hexafluoro-isopropanol solvent to prepare a solution B;
Step 3: dispersing a copper source and organic ligands in a polar solvent of 10-100 mmol/mL, stirring to form a mixed system, subjecting the mixed system to a coordination reaction, separating the reaction product by centrifugation or filtration after the reaction is completed, washing with the solvent for 2-5 times, and drying in vacuum to obtain a copper-based metal organic framework material;
Step 4: dispersing vascular endothelial growth factor in a bovine serum albumin solution to prepare a solution C;
Step 5: adding the solution B, the copper-based metal organic framework powder and the solution C successively into the solution A to prepare an electrospinning solution;
Step 6: conducting an electrospinning process according to preset parameters;
Step 7: taking out collected sample, drying and storing at a low temperature.

In step 1, the tetrahydrofuran is mixed with the N,N-dimethylformamide in a volume ratio of 1:2 to 2:1, and the mass concentration of the polyurethane in the solution A is 10%-20% (g/mL).

In step 2, the mass concentration of the gelatin in the solution B is 10%-20% (g/mL).

In step 3, the molar ratio of the copper source to the organic ligands is 0.5-5, the conditions for the coordination reaction of the mixed system are 0-200° C. and 0.5-72 hours, and the conditions for the drying in vacuum are 40-120° C. and 4-24 hours.

In step 4, the mass concentration of the vascular endothelial growth factor in the solution C is 5-20 μg/mL.

In step 5, in the electrospinning solution, the volume proportion of the solution A is 85%-88%, the volume proportion of the solution B is 8%-10%, the volume proportion of the solution C is 4%-5%, and the mass concentration of the copper-based metal organic framework powder is 5%-20% (g/mL).

In step 6, the preset parameters include a propulsion rate of 0.5-2.0 mL/h, a nozzle movement speed of 40-100 mm/min, a receiving shaft rotational speed of 100-400 rpm, a receiving distance of 16-20 cm, and a voltage of 12-22 kV.

In step 7, for the drying and storing at a low temperature, the drying environment is for drying in vacuum and room temperature conditions for 48-96 hours, and the condition for storing at a low temperature is 2-10° C.

An application of the copper-based MOF-modified small-diameter polyurethane vascular graft in vascular tissue engineering or biomedical materials is provided.

In the copper-based MOF-modified small-diameter polyurethane vascular graft, copper-based MOF is used as a functional filler to be compounded with a polyurethane matrix, thus to endow the material with suitable biomechanical properties, promote the growth of endothelial cells, inhibit the excessive proliferation of smooth muscle cells and provide an antithrombotic performance. The copper-based MOF-modified small-diameter polyurethane vascular graft is applicable to clinical needs such as coronary artery bypass grafting and peripheral vascular injury repair.

The present invention has the following beneficial effects: in the copper-based MOF-modified small-diameter polyurethane vascular graft and the preparation method therefor provided by the present invention, the present invention proposes a synergistic strategy of “mechanical adaptation—biological regulation”, which is to use copper-based MOF as a core functional carrier and polyurethane as a base material to construct a new type of small-diameter vascular graft by electrospinning technology. A fiber structure with random orientation can provide excellent mechanical stability in both axial and radial directions. At the same time, the copper ions, nitric oxide and the vascular endothelial growth factor can act synergistically on platelets, endothelial cells and smooth muscle cells, i.e., can inhibit the adhesion and activation of platelets, promote the proliferation of endothelial cells, and inhibit the excessive proliferation of smooth muscle cells, thus to achieve multi-level regulation. Therefore, the multiple needs of the small-diameter vascular graft for “anticoagulation—endothelium promotion—hyperplasia inhibition” are effectively balanced. The present invention uses copper-based MOF as a drug carrier, which solves the key problem of local burst release of drug molecules and improves the biomechanical properties of the small-diameter vascular graft.

### DESCRIPTION OF DRAWINGS

FIG. 1 shows a scanning electron microscope (SEM) image of microscopic morphology and elemental energy spectra of a copper-based MOF-modified small-diameter polyurethane vascular graft and a preparation method therefor; and (a) is the SEM image of the small-diameter vascular graft, and (b)-(d) are element distribution diagrams of C, O and Cu respectively;

FIG. 2 shows biomechanical property test results of a copper-based MOF-modified small-diameter polyurethane vascular graft and a preparation method therefor; and (a) shows a sample of the small-diameter vascular graft, and (b)-(d) respectively represent post-suture holding force, burst pressure and compliance of the sample;

FIG. 3 shows a drug molecule release curve;

FIG. 4 shows a copper ion release curve;

FIG. 5 shows endothelial cell and smooth muscle cell co-culture experiment results of a copper-based MOF-modified small-diameter polyurethane vascular graft and a preparation method therefor; and (a)-(c) are observation results for 6 hours, 12 hours and 24 hours respectively.

### DETAILED DESCRIPTION

Specific embodiments of the present invention are further described below in combination with the drawings and the technical solution.

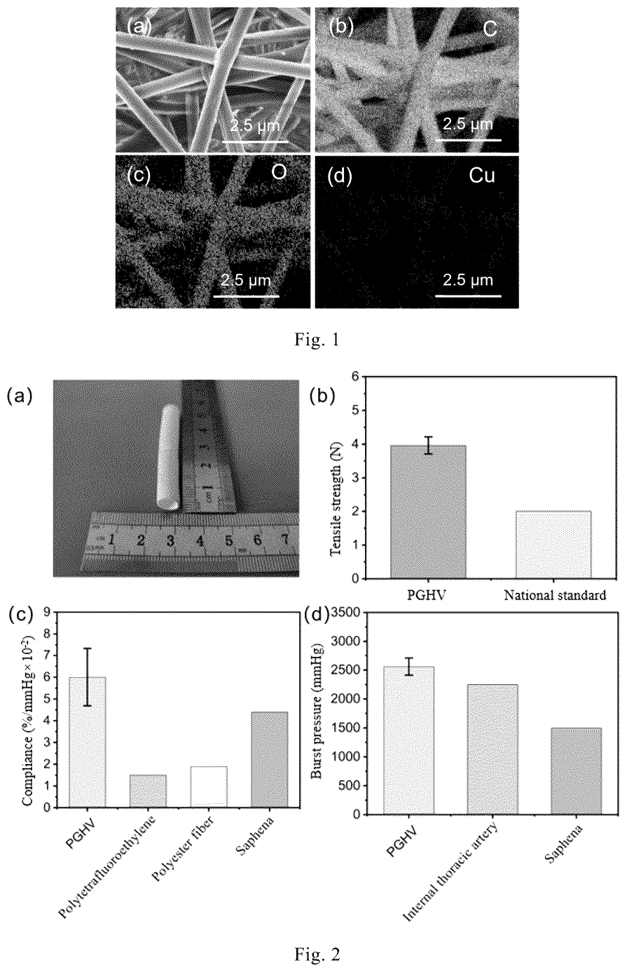

Referring to FIG. 1 to FIG. 5, the present invention provides a copper-based MOF-modified small-diameter polyurethane vascular graft, comprising a copper-based metal organic framework, polyurethane and drug molecules. The copper-based metal organic framework of the present invention is self-assembled by metal centers and organic ligands through coordination bonds to form a porous crystal structure. Each metal center is a copper ion or a copper cluster provided by a copper source, and the copper source is selected from at least one of an inorganic acid salt, an organic acid salt, an oxide and a halide of copper; each organic ligand is an organic compound with at least two coordination loci, and the coordination loci are selected from at least one of carboxyl (—COOH), amino (—NH₂), hydroxyl (—OH), pyridyl (—C₅H₄N), imidazolyl (—C₃H₃N₂) or triazolyl (—C₂H₂N₃); and the drug molecules loaded are vascular endothelial growth factor. A preparation method for the copper-based MOF-modified small-diameter polyurethane vascular graft comprises:

### Embodiment 1

Step 1: mixing tetrahydrofuran with N,N-dimethylformamide in a volume ratio, and adding polyurethane to prepare a solution A.

In the present embodiment, the tetrahydrofuran is mixed with the N,N-dimethylformamide in a volume ratio of 2:1 to a volume of 10 mL, and 1.4 g of polyurethane particles are added and stirred evenly to obtain the solution A.

Step 2: adding gelatin into a hexafluoro-isopropanol solvent to prepare a solution B.

In the present embodiment, 0.28 g of gelatin is added into 2 mL of hexafluoro-isopropanol solvent to prepare the solution B.

Step 3: after the aqueous solution of Cu(NO₃)₂·3H₂O is completely reacted with the alcohol solution of trimesic acid in a hydrothermal environment, obtaining a HKUST-1 powder by centrifugal drying.

In the present embodiment, 0.84 g of Cu(NO₃)₂·3H₂O is dissolved in 24 mL of pure water, 1.75 g of trimesic acid is dissolved in 24 mL of anhydrous ethanol, then the trimesic acid solution is dripped into the Cu(NO₃)₂·3H₂O solution to form a uniform mixed system, the mixed system is subjected to a reaction at 120° C. for 14 hours and centrifugal washing with the solvent for 6 times to obtain a centrifuged product, and the centrifuged product is dried in vacuum at 80° C. for 12 hours to obtain the HKUST-1 powder.

Step 4: dispersing vascular endothelial growth factor in a bovine serum albumin solution to prepare a solution C.

In the present embodiment, 10 μg of vascular endothelial growth factor is dissolved in 1 mL of 5 mg/mL bovine serum albumin solution to prepare a 10 μg/mL solution C.

Step 5: adding the solution B, the copper-based metal organic framework powder and the solution C successively into the solution A.

In the present embodiment, 1 mL of the solution B, 0.14 g of the copper-based metal organic framework powder and 0.5 mL of the solution C are added successively.

Step 6: conducting an electrospinning process according to preset parameters.

In the present embodiment, the preset parameters include a propulsion rate of 1.0 mL/h, a nozzle movement speed of 50 mm/min, a receiving shaft rotational speed of 200 rpm, a receiving distance of 18 cm, and a voltage of 16 kV.

Step 7: taking out collected sample, drying and storing at a low temperature.

In the present embodiment, for the drying and storing at a low temperature, the drying environment is for drying in vacuum and room temperature conditions for 72 hours, and the condition for storing at a low temperature is 4° C.

### Embodiment 2

Step 1: mixing tetrahydrofuran with N,N-dimethylformamide in a volume ratio, and adding polyurethane to prepare a solution A.

In the present embodiment, the tetrahydrofuran is mixed with the N,N-dimethylformamide in a volume ratio of 1:1 to a volume of 10 mL, and 1.4 g of polyurethane particles are added and stirred evenly to obtain the solution A.

Step 2: adding gelatin into a hexafluoro-isopropanol solvent to prepare a solution B.

In the present embodiment, 0.28 g of gelatin is added into 2 mL of hexafluoro-isopropanol solvent to prepare the solution B.

Step 3: dissolving copper chloride and 2-methylimidazole (containing imidazolyl) in methanol, stirring at room temperature to obtain a precipitate, washing the precipitate with methanol, and drying the precipitate in vacuum to obtain Cu-ZIF-L.

In the present embodiment, 1 mmol of copper chloride and 2 mmol of 2-methylimidazole (containing imidazolyl) are dissolved in 20 mL methanol and stirred at room temperature for 1 hour, and a white precipitate is generated. The precipitate is filtered, washed with methanol for 2 times, and dried in vacuum at 80° C. for 6 hours to obtain a white crystal Cu-ZIF-L.

Step 4: dispersing vascular endothelial growth factor in a bovine serum albumin solution to prepare a solution C.

In the present embodiment, 10 μg of vascular endothelial growth factor is dissolved in 1 mL of 5 mg/mL bovine serum albumin solution to prepare a 10 μg/mL solution C.

Step 5: adding the solution B, the copper-based metal organic framework powder and the solution C successively into the solution A.

In the present embodiment, 1 mL of the solution B, 0.14 g of the copper-based metal organic framework powder and 0.5 mL of the solution C are added successively.

Step 6: conducting an electrospinning process according to preset parameters.

In the present embodiment, the preset parameters include a propulsion rate of 1.0 mL/h, a nozzle movement speed of 50 mm/min, a receiving shaft rotational speed of 200 rpm, a receiving distance of 20 cm, and a voltage of 16 kV.

Step 7: taking out collected sample, drying and storing at a low temperature.

In the present embodiment, for the drying and storing at a low temperature, the drying environment is for drying in vacuum and room temperature conditions for 72 hours, and the condition for storing at a low temperature is 4° C.

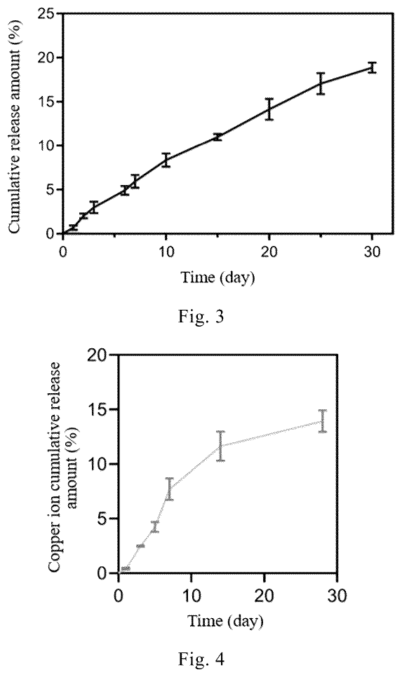

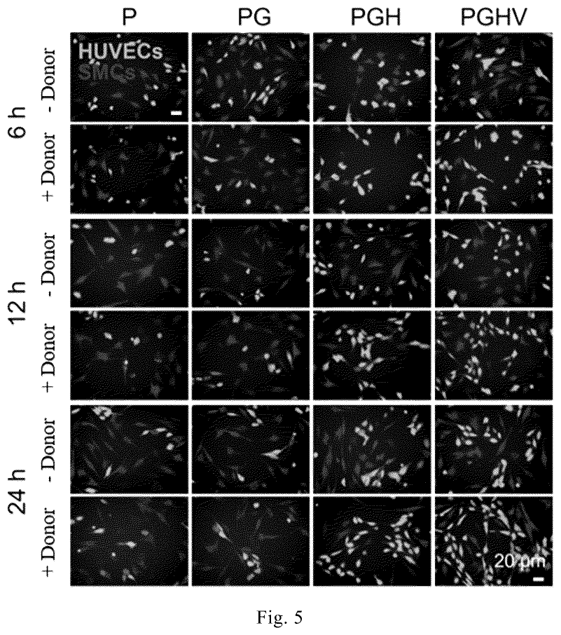

A product prepared by the present invention has the following characteristics:
Sustained-release performance: drug release>30 days, copper ion sustained-release>28 days (without sudden release);
Biocompatibility: the product can promote the adhesion and proliferation of human aortic endothelial cells, and inhibit the adhesion and proliferation of human aortic smooth muscle cells (optimal in a PGHV group);
Morphological characterization, as shown in FIG. 1: the fiber morphology is uniform, with an average fiber diameter of 0.58±0.003 μm and an average fiber pore size of 8.926±0.060 μm;
Biomechanical properties, as shown in FIG. 2: the small-diameter vascular graft prepared has a compliance value of 6.00±1.32%/100 mmHg, a burst pressure of as high as 2558.5±149.20 mmHg, and a suture holding force of 3.96±0.25 N;
Sustained-release performance, as shown in FIG. 3: the vascular endothelial growth factor is continuously released for 30 days without sudden release;
Cu²⁺ sustained-release, as shown in FIG. 4: Cu²⁺ is stably released within 28 days without sudden release;
Biocompatibility, as shown in FIG. 5: the HAECs/HASMCs ratio after 24 hours is 4.72.

To sum up, the present invention uses the copper-based metal organic framework as a functional carrier and combines electrospinning technology to solve the problem of short drug loading activity cycles. The integration of dynamic mechanical properties and biological functions provides a new paradigm for the design of vascular tissue engineering materials and lays a foundation for future in vivo experimental verification and clinical transformation.

The above embodiments of the present invention do not limit the protection scope of the present invention.

1. A copper-based MOF-modified small-diameter polyurethane vascular graft, comprising a copper-based metal organic framework, polyurethane and drug molecules, wherein the polyurethane is used as a base material for the small-diameter polyurethane vascular graft, and the drug molecules are loaded on the copper-based metal organic framework based on a porous structure thereof to modify the surface of the small-diameter polyurethane vascular graft, thus to endow the small-diameter polyurethane vascular graft with the multi-level micro-regulation functions of “anti-thrombosis-endothelium promotion-hyperplasia inhibition”.

2. The copper-based MOF-modified small-diameter polyurethane vascular graft according to claim 1, wherein the copper-based metal organic framework is self-assembled by metal centers and organic ligands through coordination bonds to form a porous crystal structure.

3. The copper-based MOF-modified small-diameter polyurethane vascular graft according to claim 2, wherein each metal center is a copper ion or a copper cluster provided by a copper source, and the copper source is selected from at least one of an inorganic acid salt, an organic acid salt, an oxide and a halide of copper; and each organic ligand is an organic compound with at least two coordination loci, and the coordination loci are selected from at least one of carboxyl, amino, hydroxyl, pyridyl, imidazolyl or triazolyl.

4. The copper-based MOF-modified small-diameter polyurethane vascular graft according to claim 3, wherein the drug molecules are vascular endothelial growth factor.

5. A preparation method for the copper-based MOF-modified small-diameter polyurethane vascular graft according to claim 1, comprising the following steps:
step 1: mixing tetrahydrofuran with N,N-dimethylformamide in a volume ratio, and adding polyurethane to prepare a solution A;
step 2: adding gelatin into a hexafluoro-isopropanol solvent to prepare a solution B;
step 3: dispersing a copper source and organic ligands in a polar solvent of 10-100 mmol/mL, stirring to form a mixed system, subjecting the mixed system to a coordination reaction, separating the reaction product by centrifugation or filtration after the reaction is completed, washing with the solvent for 2-5 times, and drying in vacuum to obtain a copper-based metal organic framework material;
step 4: dispersing vascular endothelial growth factor in a bovine serum albumin solution to prepare a solution C;
step 5: adding the solution B, the copper-based metal organic framework powder and the solution C successively into the solution A to prepare an electrospinning solution;
step 6: conducting an electrospinning process according to preset parameters;
step 7: taking out collected sample, drying and storing at a low temperature.

6. The preparation method according to claim 5, wherein
in step 1, the tetrahydrofuran is mixed with the N, N-dimethylformamide in a volume ratio of 1:2 to 2:1, and the mass concentration of the polyurethane in the solution A is 10%-20% (g/mL);
in step 2, the mass concentration of the gelatin in the solution B is 10%-20% (g/mL);
in step 4, the mass concentration of the vascular endothelial growth factor in the solution C is 5-20 μg/mL;
in step 5, in the electrospinning solution, the volume proportion of the solution A is 85%-88%, the volume proportion of the solution B is 8%-10%, the volume proportion of the solution C is 4%-5%, and the mass concentration of the copper-based metal organic framework powder is 5%-20% (g/mL).

7. The preparation method according to claim 6, wherein
in step 3, the molar ratio of the copper source to the organic ligands is 0.5-5, the conditions for the coordination reaction of the mixed system are 0-200° C. and 0.5-72 hours, and the conditions for the drying in vacuum are 40-120° C. and 4 -24 hours;
in step 6, the preset parameters include a propulsion rate of 0.5-2.0 mL/h, a nozzle movement speed of 40-100 mm/min, a receiving shaft rotational speed of 100-400 rpm, a receiving distance of 16-20 cm, and a voltage of 12-22 kV;
in step 7, for the drying and storing at a low temperature, the drying environment is for drying in vacuum and room temperature conditions for 48-96 hours, and the condition for storing at a low temperature is 2-10° C.

8. An application of the copper-based MOF-modified small-diameter polyurethane vascular graft in vascular tissue engineering or biomedical materials.
